(19) United States
(12) Patent Application Publication
(10) Pub. No.: US 2011/0269731 A1
(43) Pub. Date: Nov. 3, 2011

(54) NANOPARTICLES OF BETA-LACTAM DERIVATIVES

(76) Inventors: Patrick Couvreur, Villebon-sur-yvette (FR); Didier Demaele, Fresnes (FR); Fatima Zouhiri, Chatenay-malabry (FR)

(21) Appl. No.: 13/126,756

(22) PCT Filed: Oct. 28, 2009
(86) PCT No.: PCT/IB09/54780

§ 371 Date: Jul. 19, 2011

(30) Foreign Application Priority Data
Oct. 29, 2008 (FR) 0857355

(51) Int. Cl. A61K 31/43; C07D 499/48; A61P 31/04; B82Y 5/00; B82Y 99/00; B82Y 40/00

(52) U.S. Cl. 514/198; 540/336; 977/773; 977/900; 977/797; 977/915

(57) ABSTRACT

The present invention relates to a complex made up of at least one beta-lactam molecule covalently bonded to at least one hydrocarbon radical including at least 18 carbon atoms and containing at least one unit of 2-methyl-buta-2-ene, to nanoparticles of said complexes, and to a method for preparing same, said complex and/or said nanoparticles optionally being in the form of a lyophilisate. The present invention also relates to a pharmaceutical composition including at least said complex and/or said nanoparticles. The invention finally relates to said complex and/or to said nanoparticles for the treatment and/or prevention of bacterial infections, in particular caused by strains that are sensitive to beta-lactams.

The present invention aims to propose novel beta-lactam derivatives, in particular in a water-dispersible nanoparticulate form, compositions containing same, and therapeutic uses thereof.

Beta-lactams represent a family of bactericidal antibiotics of which the basic structure is the beta-lactam ring. Several subfamilies are distinguished, namely carbacephems, carbapenems, cephalosporins, cephamycins, monobactams, oxacephems, or else penicillins.

Antibiotics of this type, which are among the most well-tolerated by the body, are the most widely used.

However, their use comes up against various difficulties, in particular in terms of efficacy, spectrum of action and bioavailability.

First of all, the efficacy of beta-lactams is reduced by the appearance of resistance phenomena. More specifically, genetic modifications can allow certain bacterial strains to escape the action of these antibiotics. A classic example is the appearance of a gene responsible for the production of an enzyme, beta-lactamase, which as it happens inactivates beta-lactams. Nine Staphylococcus aureus out of ten have this defensive weapon.

In order to counter this resistance phenomenon, it is generally necessary to substantially modify the structure of conventional beta-lactams, retaining only their lactam nucleus as basic structure.

Thus, WO 2008/0332478 describes the employment of N-thiolated beta-lactam derivatives for their use on the methicillin-resistant S. aureus strain. However, chemical modifications of this type are generally long and expensive.

WO 2005/110407 describes, for its part, the employment of cyclic lactam derivatives, in the form of a nanoparticulate dispersion in a liquid medium, comprising a stabilizing agent adsorbed at the surface of the nanoparticles and, optionally, a surfactant. However, the stabilizers are generally of the type to cause adverse effects, in particular in terms of toxicity.

In addition to these resistance phenomena, beta-lactams have a limited spectrum of action. Thus, by virtue of their mode of action, some prove to be completely ineffective against intracellular infections, which are often opportunistic in nature.

This is because beta-lactams behave as inhibitors of the enzymes responsible for the synthesis of peptidoglycan, a major constituent of the bacterial cell wall, and do not, strictly speaking, penetrate into the bacterium, but join up with their target on the inner face of the wall (periplasmic space). This access, which is direct for gram-positive (+) bacteria, is gained via the porins in the outer membrane for gram-negative (−) bacteria. By way of example of this type of enzyme, mention may be made of PBP1 (for Penicillin Binding Protein type 1), which controls bacterial elongation and the inhibition of which causes cell lysis, or else PBP2 which controls the shape of the bacterium and the inhibition of which leads to the formation of filamentous bacteria. In the presence of beta-lactam, such enzymes exert their hydrolytic action on the antibiotic molecule and result in the formation of an enzyme-product complex which does not dissociate since the enzyme is covalently bonded to the product. This mechanism, in which the enzyme itself catalyzes the conversion of the antibiotic to a highly reactive compound which binds to the enzyme irreversibly, is called suicide inhibition.

Thus, many intracellular infections cannot be treated with beta-lactam antibiotics. This is because, since these antibiotics are acidic in nature, they are ionized at physiological pH and therefore diffuse poorly at the intracellular level. In particular, they are incapable of penetrating into the deep intracellular compartments, namely the endosomes/lysosomes, which are sites of many intracellular infections. These infections are therefore resistant to conventional antibiotic treatment.

The present invention aims precisely to overcome the above-mentioned drawbacks.

More particularly, the inventors have demonstrated that it is found to be possible to formulate beta-lactams in the form of nanoparticles in suspension in an aqueous medium, and of small size, in particular compatible with administration by injection or oral administration, with the proviso that they are covalently coupled to at least one hydrocarbon-based radical of squalene nature.

In addition, they have noted that the coupling of beta-lactam derivatives to at least one hydrocarbon-based radical according to the invention advantageously makes it possible to improve the intracellular penetration of the antibiotic, either because said hydrocarbon-based derivative confers greater lipophilicity on the beta-lactams and therefore better diffusibility properties, or owing to the particulate nature of the beta-lactam/hydrocarbon-based derivative complex (or conjugate) which promotes uptake by endocytosis, resulting in an intra-endosomal or intra-lysosomal localization of the antibiotic.

Thus, according to a first aspect, the present invention relates to a complex made up of at least one beta-lactam molecule covalently coupled to at least one hydrocarbon-based radical comprising at least 18 carbon atoms and containing at least one unit represented by the formula which follows;

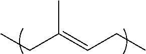

also known as 2-methylbut-2-ene.

According to another subject, the invention is directed toward a complex as defined above, in which the hydrocarbon-based compound comprises from 18 to 40 carbon atoms, preferably from 18 to 32 carbon atoms.

A subject of the present invention is a complex as defined above, in which the hydrocarbon-based radical is represented by the radical of formula (I), as defined hereinafter.

Advantageously, the two entities fowling the complex defined above are coupled by means of a covalent bond of ester, ether, thioether, disulfide, phosphate or amide type, and preferably amide type.

Another subject of the present invention is directed toward nanoparticles of a complex as described above.

Advantageously, the average size of these nanoparticles ranges from 30 to 500 nm, in particular from 50 to 250 nm, or even from 100 to 400 nm.

Nanoparticles of antibiotics of the beta-lactam family, which use synthetic polymers, such as polyacrylate or a derivative thereof, are known (TUROS et al., Bioorganic and Medicinal Chemistry Letters, Vol. 17, No. 1, 22 Dec. 2006, pp. 53-56; TUROS et al., Bioorganic and Chemistry Letters, Vol. 17, No. 12, 15 Jun. 2007, pp. 3468-3472 and BALLAND et al., The Journal of Antimicrobial Chemotherapy Vol. 37 No. 1. January 1996, pp. 105-115).

The documents WO 2006/090029 and Couvreur et al., Nano. Letters, Vol. 6, Nov. 1, 2006, pp. 2544-48, for their part, describe the fact that squalene, which is a lipid molecule of natural origin, covalently coupled to gemcitabine is found to be capable of spontaneously forming nanoparticles of about a hundred nanometers in an aqueous medium. However, this active compound is clearly very different from the active agents under consideration according to the present invention.

As indicated above, the formulation of the therapeutic active agents under consideration according to the present invention, in the form of nanoparticles in accordance with the present invention, constitutes an advantageous alternative with regard to the formulations that already exist, in several respects.

First of all, as indicated above, the nanoparticulate foam of the beta-lactams advantageously makes it possible to increase their bioavailability, in particular their intracellular bioavailability, which represents an advantage for the treatment of intracellular, in particular opportunistic, infections which are often resistant to conventional antibiotics.

What is more, this improvement in bioavailability advantageously allows a better determination of the doses to be used.

Finally, the complexes and/or nanoparticles according to the present invention are advantageously compatible with any mode of administration.

The present invention also relates to a method for preparing said nanoparticles, comprising at least the dispersion of the complex according to the present invention in at least one organic solvent, at a concentration sufficient to obtain, when the resulting mixture is added, with stirring, to an aqueous phase, the instantaneous formation of nanoparticles of said complex in suspension in said aqueous phase, and, where appropriate, the isolation of said nanoparticles.

Advantageously, said method may also optionally comprise a lyophilization step, in particular suitable for being able to formulate in solid form.

Thus, the present invention also extends to a lyophilisate comprising at least one complex and/or at least nanoparticles as described above.

The present invention is also directed toward a pharmaceutical composition, in particular a medicament, comprising at least one complex and/or nanoparticles, said complexes and/or nanoparticles, optionally in the form of a lyophilisate, as described above, in combination with at least one pharmaceutically acceptable vehicle.

According to another subject, the present invention relates to the use of a complex and/or the nanoparticles as defined above, optionally in the form of a lyophilisate as defined above, for preparing a pharmaceutical composition intended for treating and/or preventing bacterial infections, in particular caused by beta-lactam-sensitive strains.

In other words, the present invention is directed toward a complex and/or nanoparticles, optionally in the form of a lyophilisate, as defined above, for the treatment and/or prevention of bacterial infections, in particular caused by beta-lactam-sensitive strains.

By way of bacteria targeted according to the present invention, responsible for infections, mention may, for example, be made of gram-negative (−) or -positive (+) bacteria.

By way of gram (−) bacteria, mention may, for example, be made of bacteria of the Bacteroides genus, such as B. fragilis, Helicobacter pylori, Campylobacter, Leptospira, Borrelia, Treponema, Fusobacterium, Enterobacter spp., Escherichia coli, Haemophilus influenzae, Klebsiella species, Morganella morganii, Neisseria gonorrhoeae, Proteus mirabilis, Proteus vulgaris, Providencia rettgeri, Pseudomonas spp., such as P. aeruginosa and Serratia marcescens.

By way of gram (+) bacteria, mention may, for example, be made of Enterococcus faecalis, Peptococcus spp., Peptostreptococcus spp., Listeria, Clostridium perfringens and salmonellae, in particular Salmonella typhimurium.

The most common primary infections are, for example, sore throats, ear infections and intestinal ailments. They may also involve gastroenteritis, urinary infections, meningitis or septicemia.

Thus, the complexes and/or nanoparticles prove to be particularly useful for the treatment and/or prevention of infections caused by bacteria of the pneumococcal (pneumonia, ear infection, meningitis), streptococcal (sore throat), meningococci (meningitis), salmonella, treponema (syphilis), Listeria (listeriosis), Clostridium perfringens, Helicobacter pylori or else Escherichia coli type.

More recently, it has been discovered that β-lactam derivatives can prove to be useful for inducing tumor cell apoptosis (Synthetic Beta-Lactam antibiotics as a selective Poreast cancer prevention and treatment—Dr. Q. Ping Dou, Wayne State University; Annual Summary, Mar. 24, 2004-Mar. 23, 2005 and WO 2004/100888).

Thus, the present invention is also directed toward a complex and/or nanoparticles optionally in the form of a lyophilisate, as defined above, for the prevention or adjuvant treatment of cancers.

Hydrocarbon-Based Compound or Radical Having a Squalene Structure

For the purpose of the present invention, a compound or radical having a squalene or squalenoyl structure is a compound or radical comprising at least one 2-methylbut-2-ene unit, as defined above.

More specifically, a hydrocarbon-based compound or radical having a squalene or squalenoyl structure comprises at least 18 carbon atoms and contains at least one 2-methylbut-2-ene unit, like a squalene radical.

It should be noted that, in the present invention, reference is made, as appropriate, to a “compound” or “radical” having a squalene or squalenoyl structure. The term “compound” is intended more specifically to define a squalenoyl derivative, which, when it reacts with a molecule of active agent, forms a complex, whereas the term “radical” defines more specifically the squalene or squalenoyl part of the complex formed.

For the purpose of the present invention, a hydrocarbon-based radical having a squalene structure can be represented by formula (I) which follows:

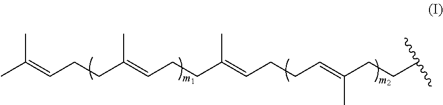

in which:
m₁=1, 2, 3, 4, 5 or 6;
m₂=0, 1, 2, 3, 4, 5 or 6; and

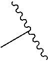

represents the bond toward the molecule, derived from beta-lactam, it being understood that, when m₂ represents 0, then m₁ represents at least 2.

More specifically, when reference is made to the squalenoyl compound or a derivative thereof, a starting entity having served for the coupling, this compound or a derivative thereof can be represented by the compound of formula (Ia):

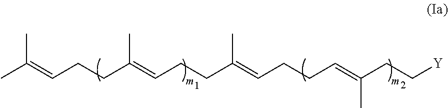

in which:
Y represents a hydrogen atom, or an -L-X′ group in which X′ represents a function of alcohols, carboxylic acid, thiol, phosphate, amine, carboxamide or ketone type and L represents a single covalent bond or a C₁-C₄ alkylene group; and
m₁ and m₂ are as defined for the radical of formula (I).

The hydrocarbon-based radical comprises at least 18 carbon atoms, in particular from 18 to 40 carbon atoms, and preferably from 18 to 32 carbon atoms.

More specifically, a compound which is of use for the formation of a complex according to the present invention is squalene (also known as spiracene or sirprene), which is an essential intermediate of cholesterol biosynthesis. Its chemical name is: (E)-2,6,10,15,19,23-hexamethyl-2,6,10,14,18,22-tetracosahexene and it can be represented by the formula which follows:

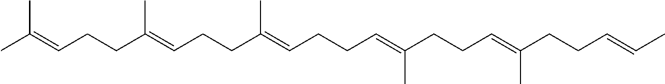

According to one preferred embodiment of the invention, the squalene derivative present in a complex according to the present invention is a radical of formula (I) in which m₁=1 and m₂=2.

Advantageously, said complex is a radical of formula (I) in which m₁=1 and m₂=3.

According to one preferred embodiment of the present invention, the squalene derivative present in a complex according to the present invention is a radical of formula (I′) which follows, corresponding to a radical of formula (I) above in which m₂=0:

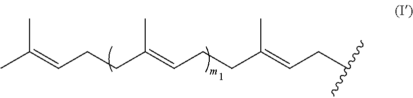

In this case, m₁=2, 3, 4, 5 or 6.

By way of illustration of the hydrocarbon-based compounds capable of forming a complex according to the present invention, mention may more particularly be made of squalenic acid and derivatives thereof, such as 1,1′,2-trisnorsqualenic acid, squalene acetic acid, 1,1′,2-trisnorsqualenyloxyacetic acid, 1,1′,2-trisnorsqualenylaminoacetic acid, 1′,2-trisnorsqualenylsulfanylacetic acid, squalenol or squalene amine.

A complex according to the present invention comprises at least one hydrocarbon-based radical represented by a radical of formula (I) as defined above.

Alternatively, a complex according to the present invention comprises at least two hydrocarbon-based radicals as defined above and in particular at least two hydrocarbon-based radicals represented by a radical of formula (I) as defined above.

In particular, a complex according to the present invention may contain at least one radical derived from a 1,1′,2-trisnorsqualenic acid molecule.

As noted by the inventors, an abovementioned hydrocarbon-based compound of squalenoyl type spontaneously manifests, when it is placed in the presence of a polar medium, and more particularly water, a compacted conformation.

Unexpectedly, the inventors have noted that this ability remains when such a compound is associated with, in particular covalently bonded to, a beta-lactam molecule. This results in the creation of a compacted architecture in the form of nanoparticles, in which there is at least partly a beta-lactam molecular entity and at least one hydrocarbon-based radical.

The beta-lactam molecule may in fact be only partly or be totally in the compacted state in the nanoparticles formed.

Generally, at least one abovementioned hydrocarbon-based radical is covalently bonded to a beta-lactam molecule. However, the number of molecules of hydrocarbon-based derivative capable of interacting with said molecule may be greater than 1.

Beta-Lactam

As indicated above, “beta-lactam” or “beta-lactam antibiotics” or “derivatives thereof” is intended to mean any antibiotic which contains a beta-lactam nucleus in its molecular structure.

More specifically, this beta-lactam nucleus can be inserted into a bicyclic structure represented by the following radical (A):

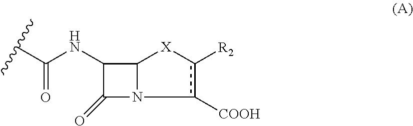

in which:

X represents a heteroatom chosen from a sulfur, an oxygen, a nitrogen or else a divalent radical —S—CH₂—, —CH₂— or —(CH₂)₂—;

[symbol] indicates the optional presence of a double bond;

R₂ represents one or two group(s) chosen independently from a hydrogen atom, a halogen atom, a hydroxyl group, a C₁-C₆ alkyl, in particular a methyl group, and a C₁-C₆ alkoxy, in particular a methoxy, said alkyl and alkoxy groups being optionally substituted with one or more halogen atoms, with one or more hydroxyl groups or with an —O—C(O)—C₁-C₆alkyl group, in particular —O—C(O)-methyl.

Thus, mention may, for example, be made of penicillins, cephalosporins, carbapenems, beta-lactamase inhibitors.

More particularly, for the purpose of the present invention, the term “beta-lactam” is intended to mean a derivative represented by formula (II) which follows:

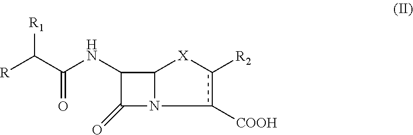

in which:
R represents an aryl group, in particular a phenyl group, an —O-phenyl group or a heteroaryl group, said groups being optionally substituted with one or more R₃ group(s);
R₃ represents a halogen atom, a hydroxyl group, a C₁-C₆ alkyl group, a C₁-C₆ alkoxy group, said alkyl and alkoxy groups being optionally substituted with one or more halogen atoms or with one or more hydroxyl groups, an —NR₄R₅ group, a —COOR₆ group or a —CONR₄R₅ group;
R₄ and R₅ represent, independently of one another, a hydrogen atom, or a C₁-C₆ alkyl group optionally substituted with one or more halogen atoms or with one or more hydroxyl groups;
R₆ represents a hydrogen atom, or a C₁-C₆ alkyl optionally substituted with one or more halogen atoms or with one or more hydroxyl groups;
R₁ represents a hydrogen atom, or a —COOR₆, —NR₄R₅ or ═N—OCH₃ group;
   X and R₂ being as defined above, for the radical of formula (A),

and the pharmaceutically acceptable salts thereof.

For the purpose of the present invention:
the term “a halogen atom” is intended to mean: a fluorine atom, chlorine atom, a bromine atom or an iodine atom;
the term “a hydroxyl group” is intended to mean an —OH group;
the term “an alkyl” is intended to mean: a linear or branched, saturated aliphatic group. By way of example, mention may be made of methyl, ethyl, propyl, isopropyl, butyl, isobutyl, sec-butyl and tert-butyl;
the term “an alkoxy” is intended to mean: an —O-alkyl radical where the alkyl group is as defined above, for example methoxy, ethoxy, propoxy, isopropoxy, butoxy, isobutoxy, sec-butoxy and tert-butoxy;
the term “an aryl group” is intended to mean: an aromatic group that may be partially unsaturated and monocyclic or bicyclic, comprising from 6 to 10 carbon atoms. By way of example of a monocycle, mention may be made of phenyl. By way of example of a bicycle, mention may be made of naphthyl;
the term “a heteroaryl group” is intended to mean: an abovementioned aryl group comprising, in addition, at least one heteroatom chosen from a nitrogen, a sulfur or an oxygen. By way of example of a monocycle, mention may be made of furanyl, thiophenyl, thienyl pyrrolyl, thiazolyl, imidazolyl, pyrazolyl, isoxazolyl, thiadiazolyl, pyridinyl, pyrimidyl and pyrazinyl. By way of example of a bicycle, mention may be made of indolyl, isoindolyl, indolizinyl, benzofuranyl, benzimidazolyl, quinolyl, isoquinolyl and phthalazyl.

Advantageously, use is made of a compound of formula (II) in which X represents a sulfur atom or an —S—CH₂— radical.

According to one particular embodiment, the beta-lactams in accordance with the invention which may be used may be represented by the compound of formula (IIa) which follows, and the salts thereof:

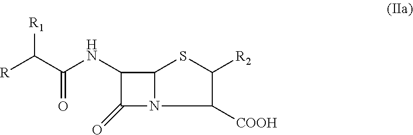

in which R and R₁ are as defined for the compound of formula (II) and R₂ is as defined for the radical of formula (A).

The penicillin family is more particularly represented by this family (IIa). Mention may in particular be made of penicillin G.

Among these compounds of formula (IIa), mention may most particularly be made of the compounds of formula (IIa) in which:

R₂ represents two C₁-C₆ alkyl groups, in particular two methyls, grafted to the rest of the molecule on the same carbon atom;

R₁ represents a hydrogen atom, an —NR₄R₅ group in which R₄ and R₅ represent a hydrogen atom, or a—COOR₆ group in which R₆ represents a hydrogen atom;

R represents a phenyl optionally substituted with one or two C₁-C₆ alkoxy, in particular methoxy, group(s).

According to another particular embodiment, the beta-lactams that may be used according to the present invention may be represented by the compound of formula (IIb) which follows, and the salts thereof:

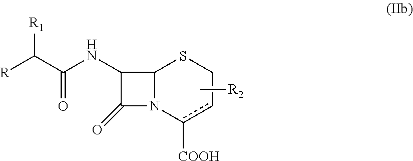

in which:

[symbol] and the R, R₁ and R₂ groups are as defined for the compound of formula (II).

The cephalosporin family is more particularly represented by this formula (IIb).

Among these compounds of formula (IIb), mention may most particularly be made of the compounds of formula (IIb) in which:

R₂ represents a C₁-C₆ alkyl group, in particular a methyl, substituted with an —O—C(O)—C₁-C₆ alkyl, in particular —O—C(O)-methyl, group;

R₁ represents an ═N—OCH₃ group;

R represents a monocyclic heteroaryl group, in particular a thiazolyl, optionally substituted with an —NR₄R₅ group in which R₄ and R₅ are as defined above and in particular represent a hydrogen atom.

The compounds of general formula (II), (IIa) or (IIb) may comprise one or more asymmetric carbons. They may therefore exist in the form of enantiomers or of diastereoisomers. These enantiomers and diastereoisomers, and also mixtures thereof, including racemic mixtures, are part of the invention.

The compounds of general formula (II), (IIa) or (IIb) may also exist in the form of atropoisomers.

The compounds of formulae mentioned above may exist in the form of bases or of addition salts with acids. Mention may, for example, be made of the corresponding sodium salts. Such addition salts are part of the invention.

These salts are advantageously prepared with pharmaceutically acceptable acids, but the salts of other acids that are of use, for example, for purifying or separating the compounds of abovementioned formulae are also part of the invention.

The compounds of general formula (II), (IIa) or (IIb) may also be in the form of hydrates or of solvates, namely in the faun of associations or combinations with one or more molecules of water or with a solvent. Such hydrates and solvates are also part of the invention.

A beta-lactam most particularly suitable for implementation of the present invention is chosen from penicillins, cephalosporins and carbapenems. Penicillins and cephalosporins are preferably used.

Among the penicillins, mention may, for example, be made of: amdinocillin, amdinocillin pivoxil, amoxicillin, ampicillin, apalcillin, aspoxicillin, azidocillin, bacampicillin, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin, oxacillin, penamecillin, penethamate hydriodide, penicillin G., penicillin G. benzathine, penicillin G. procaine, penicillin N, penicillin O, penicillin V, penimepicycline, phenethicillin potassium, piperacillin, pivampicillin, propicillin, quinacillin, sulbenicillin, sultamicillin, talampicillin, temocillin and ticarcillin.

Among the cephalosporins, mention may, for example, be made of: cefaclor, cefadroxil, cefamandole, cefatrizine, cefazedone, cefazoline, cefcapene pivoxil, cefclidine, cefdinir, cefditorene, cefepime, cefetamet, cefixime, cefimenoxime, cefodizime, cefonicid, cefoperazone, ceforanide, cefoselis, cefotaxime, cefotiam, cefozopram, cefpiramide, cefpirome, cefpodoxime proxetil, cefprozil, cefroxadine, cefsulodine, ceftazidime, cefteram, ceftezole, ceftibuten, ceftizoxime, ceftriaxone, cefuroxime, cefuzonam, cephacetrile sodium, cephalexin, cephaloglycine, cephaloridine, cephalosporin C, cephalothin, cephapirin sodium, cephradrine and pivcephalexin.

Among the carbapenems, mention may, for example, be made of: biapenem, ertapenem, fropenem, imipenem, meropenem and panipenem.

Advantageously, according to the present invention, amoxicillin, ampicillin, penicillin G, cefotaxime, floxacillin, methicillin, dicloxacillin, carbenicillin and mezlocillin, and more particularly ampicillin, amoxicillin and penicillin G, are used in the complexes and/or nanoparticles according to the invention.

Beta-Lactam/Hydrocarbon-Based Radical Complex

The conjugation of a beta-lactam molecule with a hydrocarbon-based compound in accordance with the invention, and more particularly with squalenic acid, confers on the beta-lactam molecule physicochemcial characteristics sufficient to give it an ability to form particles by nanoprecipitation, the size of which particles is found to be compatible with any mode of administration, in particular intravenous and oral.

For the purpose of the present invention, such a conjugation results in the formation of a complex or conjugate of beta-lactam/hydrocarbon-based radical as defined above, i.e. an entity comprising a radical derived from a beta-lactam molecule, covalently bonded to a hydrocarbon-based radical as defined above. For the purpose of the present invention, the terms “complex” or “conjugate” may be used without distinction to refer to this entity.

Thus, the present invention is directed toward a complex according to the invention, characterized in that it has the ability to spontaneously organize in the form of nanoparticles when it is in the presence of an aqueous medium.

The formation of the beta-lactam/hydrocarbon-based radical complex according to the invention requires that the two entities of the complex bear functions capable of forming a covalent bond and/or a linker arm; as defined below. These functions may or may not be present on the two starting entities. If they are not present, the starting entity will have to undergo a modification, prior to the coupling reaction.

More specifically, the hydrocarbon-based compound according to the invention generally bears a function capable of reacting with a function present on the beta-lactam molecule under consideration, so as to establish a covalent linkage between the two entities, for example of ester, ether, thioether, disulfide, phosphate or amide type, thus forming a covalent complex.

Advantageously, the function may be an amide function. In this case, the hydrocarbon-based compound having a terpenic structure capable of reacting with a beta-lactam molecule or a derivative thereof so as to form the abovementioned complex is 1,1′,2-trisnorsqualenic acid or a derivative thereof, and in particular the acid chloride or the mixed anhydride with ethyl chloroformate.

According to one embodiment variant, the covalent linkage that exists between the two types of molecules can be represented by a spacer or alternatively a linker arm. Such an arm may in particular prove to be useful for increasing the force of the beta-lactam/hydrocarbon-based radical interaction or rendering the beta-lactam/hydrocarbon-based radical bond according to the invention more sensitive to the action of enzymes.

Such an arm in fact makes it possible to introduce, via each of the two ends of its backbone, the appropriate functions, i.e. functions respectively having the expected reactional affinity, one for the function present on the compound having a hydrocarbon-based structure according to the invention, and the other for the function present on the beta-lactam molecule under consideration.

It may also be envisioned that this linker arm additionally has in its backbone a labile function, which is subsequently suitable for separating the compound having a hydrocarbon-based structure from the beta-lactam molecule under consideration. It may, for example, be a peptide unit that can be recognized by an enzyme.

Units of linker arm type are well known to those skilled in the art and their use clearly falls within the competence thereof.

By way of representation of the linker arms that may be envisioned according to the invention, mention may in particular be made of the alkylene chains as defined above, (poly)amino acid units, polyol units, saccharide units, and polyethylene glycol (polyetheroxide) units.

For the purpose of the present invention:
the term “saccharide unit” is intended to mean a radical comprising at least one radical chosen from trioses (glyceraldehyde, dihydroxyacetone), tetroses (erythrose, threose, erythrulose,), pentoses (arabinose, lyxose, ribose, deoxyribose, xylose, ribulose, xylulose), hexoses (allose, altrose, galactose, glucose, gulose, idose, mannose, talose, fructose, psicose, sorbose, tagatose), heptoses (mannoheptulose, sedoheptulose), octoses (octolose, 2-keto-3-deoxymannooctonate), isonoses (sialose), and
the term “(poly)amino acid unit” is intended to mean a unit having at least one unit:

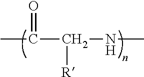

in which n is greater than or equal to 1 and R′ represents a hydrogen atom, a C₁-C₆ alkyl group, optionally substituted with one or more hydroxyls, or a C₁-C₆ alkoxy.

Thus, for the purpose of the present invention, a “covalent linkage” preferably represents a covalent bond, in particular as specified above, but also covers a covalent linkage represented by a linker arm as defined above.

Thus, a covalent complex according to the present invention can be represented by the compound of formula (III) which follows, and the salts thereof:

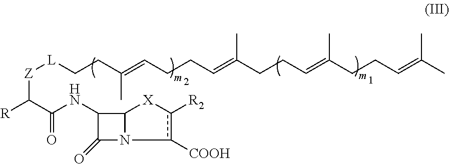

in which:
X, R and R₂ are as defined above for the compound of formula (II) and m₁ and m₂ are as defined above for the compound of formula (I); and
Z represents a covalent bond of ester, ether, thioether, disulfide, phosphate or amide type and L represents a single covalent bond or a C₁-C₄ alkylene group.

For the purpose of the present invention, the term “C₁-C₄ alkylene group” is intended to mean a divalent alkyl group that may comprise from 1 to 4 carbon atoms. By way of example, mention may be made of methylene, propylene, isopropylene and butylene.

The present invention advantageously uses a complex represented by the compound of formula (IIIa) which follows, and the salts thereof:

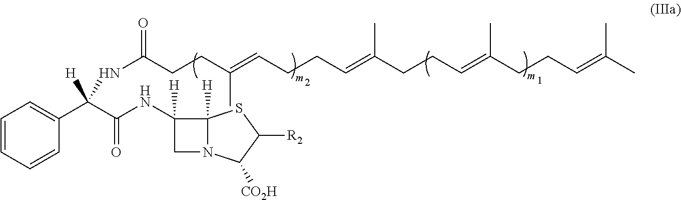

in which m₁ and m₂ are as defined above for the compound of formula (I) and R₂ is as defined for the compound of formula (II) or (IIa).

Alternatively, in the case of a beta-lactam molecule of formula (IIa), the hydrocarbon-based radical according to the invention is attached thereto via the carboxylic acid function of the compound of formula (IIa), either by means of a direct covalent bond, or by means of a bond of —Z-L- type as defined above.

A covalent complex according to the present invention can be represented by the compound of formula (IIIb) which follows, and the salts thereof:

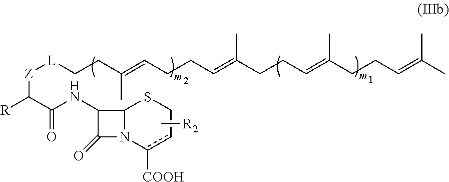

in which:

Z, L, R and R₂ are as defined for the compound of formula (III) and m₁ and m₂ are as defined for the compound of formula (I).

A subject of the present invention is therefore a complex in accordance with the invention, that may be represented by the formula (III), (IIIa) or (IIIb), as defined above.

Method for Preparing the Complex

The reaction required for establishing at least one covalent bond between at least one beta-lactam molecule under consideration and at least one hydrocarbon-based radical in accordance with the present invention can be carried out according to standard conditions, and the implementation thereof is therefore clearly part of the knowledge of those skilled in the art.

This reaction is generally carried out in solution in the presence and with an excess of at least one hydrocarbon-based compound under consideration according to the present invention relative to the beta-lactam molecule used according to the invention, for example in a proportion of two equivalents, according to the standard conditions required for interaction between the two specific functions borne by each of the two entities.

As indicated above, the establishment of the covalent bond between the two entities to be considered according to the invention requires that they bear functions capable of reacting with one another, for instance a carboxyl function with a hydroxyl function so as to form an ester bond or alternatively an amine function with a carboxyl function so as to form an amide bond.

Thus, if necessary, one or both entities, on the one hand the beta-lactam molecule and, on the other hand, the hydrocarbon-based molecule, are modified prior to the coupling reaction in order to provide them with the appropriate function in order to confer on them the reactivity necessary for the formation of a covalent bond between them. Preferably, each of the two molecules is modified in order to establish an amide bond between them. This type of modification is in particular of use in the case of a beta-lactam molecule of formula (IIa) comprising a basic nitrogenous group, such as ampicillin or amoxicillin.

Preferably, a starting hydrocarbon-based compound for the synthesis of a complex according to the invention is a squalene derivative in acid form, for instance 1,1′,2-trisnorsqualenic acid, which can be prepared according to the method described in example 1. Such a hydrocarbon-based compound will in particular be used in the case of coupling with a beta-lactam molecule of formula (IIa) comprising a basic nitrogenous group, such as ampicillin or amoxicillin.

Next, the covalent coupling of the two entities of the complex in accordance with the invention can in particular be carried, out as follows.

A complex according to the invention, in particular a compound of formula (III) described above, is obtained by condensation of a compound of formula (II) with a squalenoyl compound of formula (Ia), respectively bearing a function capable of reacting, in the presence of an organic solvent.

For example, in the compound of formula (Ia), Y may be a carboxylic acid function and in the compound of formula (II), R₁ may represent an —NH₂ group. Such an amine function may already be present on the compound of formula (II)—this is in fact the case for ampicillin or amoxicillin (as described in examples 1 and 3)—or else may be chemically formed thereon, prior to the condensation reaction. The organic solvent may, for example, be anhydrous tetrahydrofuran (THF) or dimethylformamide (DMF).

In the case where the beta-lactam molecule of formula (IIa) under consideration lacks the abovementioned amine function, i.e. in which R₁ may represent a hydrogen atom, such as penicillin G, the squalenoyl derivative capable of reacting with the carboxylic acid function (or its corresponding carboxylate form) of the abovementioned compound of formula (IIa) may, for example, be 1,1′,2-trisnorsqualenyl bromoacetate, resulting from the corresponding aldehyde, as is described in example 5.

Such adjustments in terms of the starting reactants clearly fall within the competence of those skilled in the art.

Nanoparticles According to the Invention

As specified above, the covalent coupling of at least one beta-lactam molecule under consideration according to the invention with at least one hydrocarbon-based compound for the purpose of the invention is of a nature to give the beta-lactam molecule thus complexed an ability to become organized in a compacted form in a polar solvent medium, thus leading to the formation of nanoparticles.

In general, the nanoparticles thus obtained have a mean size ranging from 30 to 500 nm, and in particular from 50 to 250 um, or even from 100 to 400 nm, measured by light scattering using a Coulter® N4MD nanosizer from Coulter Electronics, Hialeah, USA.

A subject of the invention is directed toward nanoparticles in accordance with the invention, the mean size of which ranges from 30 to 500 nm, in particular from 50 to 250 nm, or even from 100 to 400 nm.

Advantageously, the nanoparticles according to the present invention, in particular in the form of a lyophilisate, are particularly advantageous for oral administration.

Method for Preparing the Nanoparticles

The formation of the nanoparticles from the complexes described above can be carried out according to conventional techniques, insofar as they involve bringing the complex into contact with an aqueous medium under conditions suitable for its agglomeration in the form of nanoparticles. This may in particular involve methods referred to as nanoprecipitation or emulsion/solvent evaporation.

The nanoparticles according to the present invention may advantageously be obtained in the following way.

Preliminarily, a beta-lactam/hydrocarbon-based compound complex is formed by coupling at least one hydrocarbon-based compound according to the invention to at least one beta-lactam molecule according to the invention, as described above.

Said complex obtained is then dispersed in at least one organic solvent (for example an alcohol such as ethanol, or acetone) at a concentration sufficient to obtain, when the resulting mixture is added, with stirring, and generally dropwise, to an aqueous phase, the instantaneous formation of nanoparticles according to the invention in suspension in said aqueous phase. Where appropriate, said nanoparticles are isolated according to techniques well known to those skilled in the art.

The reaction can generally be carried out at ambient temperature. Irrespective of its value, the reaction temperature should not affect the activity of the beta-lactam molecule under consideration. The method for preparing the nanoparticles according to the invention is particularly advantageous since it does not require the obligatory presence of surfactants.

Advantageously, the formation of nanoparticles according to the present invention does not require the use of surfactants.

This property is particularly beneficial since a large number of surfactants do not prove to be compatible with an in vivo application.

However, it is understood that the use of surfactants, generally advantageously free of any toxicity, can be envisioned in the context of the invention. Surfactants of this type may, moreover, make it possible to obtain even smaller sizes during the formation of nanoparticles. By way of nonlimiting illustration of surfactants of this type which can be used in the present invention, mention may in particular be made of polyoxyethylene-polyoxypropylene copolymers, phospholipid derivatives and lipophilic derivatives of polyethylene glycol.

As a lipophilic derivative for polyethylene glycol, mention may, for example, be made of polyethylene glycol cholesterol. As examples of polyoxyethylene-polyoxypropylene block copolymers, mention may particularly be made of polyoxyethylene-polyoxypropylene-polyoxyethylene triblock copolymers, also known as Poloxamers®, Pluronics® or synperonics, and which are sold in particular by the company BASF.

Poloxamines, which are related to these families of copolymers, and which consist of hydrophobic segments (based on polyoxypropylene), hydrophilic segments (based on polyoxyethylene) and a central part deriving from the ethylenediamine unit, can also be used.

The nanoparticles according to the invention are, of course, capable of bearing, at the surface, a multitude of reactive functions, such as hydroxyl or amine functions, for example. It is therefore possible to envision attaching all sorts of molecules to these functions, in particular via covalent bonds.

By way of nonlimiting illustration of molecules of this type which are capable of being combined with the nanoparticles, mention may in particular be made of molecules of label type, compounds capable of performing a targeting function, and also any compound that is capable of conferring particular pharmacokinetic characteristics thereon. With regard to the latter aspect, it may thus be envisioned to attach, at the surface of these nanoparticles, lipophilic derivatives of polyethylene glycol, for instance the polyethylene glycol/cholesterol conjugate, polyethylene glycol phosphatidylethanolamine, or better still polyethylene glycol/squalene. Specifically, given the natural affinity of squalene residues for one another, the polyethylene glycol/squalene conjugate associates, in the case in point, with the nanoparticles according to the invention, and thus results in the formation of nanoparticles surface-coated with polyethylene glycol. Moreover, and as mentioned above, the polyethylene glycol/squalene conjugate advantageously acts, during the process of formation of the nanoparticles according to the invention, as a surfactant owing to its amphiphilic behavior and therefore stabilizes the colloidal suspension, thus reducing the size of the nanoparticles formed. A surface coating based on such compounds, and in particular polyethylene glycol or the polyethylene glycol/cholesterol conjugate or the polyethylene glycol/squalene conjugate, is in fact advantageous for imparting increased vascular remanence owing to a significant reduction in uptake of the nanoparticles by liver macrophages.

According to one advantageous embodiment, the nanoparticles according to the invention are formulated in the form of an aqueous dispersion.

According to another particular embodiment, this aqueous dispersion contains less than 5% by weight, or even less than 2% by weight, and more particularly is devoid of surfactant or the like, for instance polyethylene glycols and polyglycerol, and derivatives thereof, such as the esters for example.

According to another advantageous embodiment, this aqueous dispersion contains less than 5% by weight, or even less than 2% by weight of C₂ to C₄ alcohol, for instance ethanol.

Thus, the formulation, in an aqueous medium, of the beta-lactam under consideration by means of squalenic acid in the form of water-dispersible nanoparticles advantageously makes it possible to obtain a suspension of nanoparticles without any additive other than the 5% dextrose necessary to make the injectable suspension isotonic.

According to another advantageous embodiment, the nanoparticles according to the invention are in the form of a lyophilisate.

As indicated above, the present invention is also directed toward the use of at least one nanoparticle according to the invention in pharmaceutical compositions.

Another aspect of the invention therefore relates to a pharmaceutical composition comprising, as active material, at least one complex in accordance with the present invention, in particular in the form of nanoparticles. The complexes in accordance with the present invention may be combined therein with at least one pharmaceutically acceptable vehicle.

By way of examples of pharmaceutical formulations compatible with the compositions according to the invention, mention may in particular be made of:
intravenous injections or infusions;
saline solutions or solutions of purified water;
compositions for inhalation;
capsules, sugar-coated tablets, cachets or syrups in particular incorporating, as vehicle, water, calcium phosphate, sugars, such as lactose, dextrose or mannitol, talc, stearic acid, starch, sodium bicarbonate and/or gelatin.

When the complexes and/or nanoparticles are used as a dispersion in an aqueous solution, they may be combined with excipients such as sequestering or chelating agents, antioxidants, pH regulators and/or buffering agents.

In addition to the abovementioned compounds, the pharmaceutical compositions according to the invention may contain agents such as preservatives, wetting agents, solubilizing agents and colorants.

They may, however, contain other active agents of which it may be beneficial to take advantage from a therapeutic point of view, together with the effect of the beta-lactams.

By way of representation of these active materials that may be combined with the complexes and/or nanoparticles in accordance with the present invention, mention may in particular be made of other anticancer or cytostatic molecules or macromolecules (for example platinum salts, anthracyclines, mitotic spindle poisons, topoisomerase inhibitors, kinase inhibitors or metalloprotease inhibitors), anti-inflammatories of corticoid type (for example dexamethasone) or noncorticoid type or else molecules with immunoadjuvant activity (for example antibodies with anticancer activity), molecules with analgesic activity, such as dextropropoxyphene, tramadol, nefopan, paracetamol, acetaminophen, non-steroidal anti-inflammatory drugs (NSAIDs), including aspirin, ibuprofen, indomethacin, unefenamic acid, oxicam derivatives, coxibs (Celecoxib®, Rofecoxib®,Valdecoxib®, Parecoxib®, for example) and sulfonanilides (Nimesulide® for example).

Mention may also be made of antioxidants, such as catechins, polyphenols, flavonols, flavonones, caffeine, ascorbic acid, citric acid, tartaric acid, lecithins or natural or synthetic tocopherols.

These active materials may also be chosen from analgesics such as paracetamol, codeine or aspirin.

The formulation of the beta-lactams in the form of nanoparticles prevents any chemical condensation interaction between these two types of active agents and therefore allows them to be conditioned in the same galenical formula.

The complexes or nanoparticles in accordance with the present invention may be administered by any of the conventional routes. However, as specified above, given the small size of their particles, they can be administered intravenously in the form of an aqueous suspension and are therefore compatible with the vascular microcirculation.

For obvious reasons, the amounts of derivatives according to the invention which can be used may vary significantly depending on the method of use and the route selected for their administration.

On the other hand, for topical administration, it may be envisioned to formulate at least one complex and/or nanoparticle in accordance with the present invention in a proportion of from 0.1% to 20% by weight, or even more, relative to the total weight of the pharmaceutical formulation under consideration.

The examples which follow illustrate the present invention without it, however, being limited thereto.

The infrared spectra are obtained by measurement on a pure solid or liquid using a Fourier spectrometer (Bruker Vector® 22 Fourier Transform spectrometer). Only the significant absorptions are noted.

The optical rotations were measured using a Perkin-Elmer® 241 polarimeter. at a wavelength of 589 nm.

The ¹H and ¹³C NMR spectra were recorded using a Bruker AC® 200P spectrometer (at 200 MHz and 50 MHz, respectively, for ¹H and ¹³C) or a Bruker Avance® 300 spectrometer (at 300 MHz and 75 MHz, respectively, for ¹H and ¹³C).

The mass spectra were recorded using a Bruker Esquire-LC® instrument.

The thin layer chromatography analysis was carried out on plates pre-coated with silica 60F₂₅₄ gel (layer of 0.25 mm).

The column chromatography purifications were carried out on silica 60 gel (Merck, 230-400 mesh ASTM).

All the reactions using compounds sensitive to air or to water were carried out under a nitrogen atmosphere.

### EXAMPLE 1

### Preparation of the (N)-squalenoylampicillin (SQampi) for 3,3-dimethyl-7-oxo-6-[2-(4,8,13,17,21-penta methyldocosa-4,8,12,16,20-pentaenoylamino)-2-phenylacetylamino]-4-thia-1-azabicyclo[3.2.0]heptane-2-carboxylic acid) complex

### a) Synthesis of 1,2,2′-trisnorsqualenic acid

2.77 g (7.2 mmol) of 1,1′,2-trisnorsqualenic aldehyde (SQCHO) (Ceruti M. et al., J. Chem. Soc., Perkin Trans, 1; 2002, 1477-1486) are dissolved in 40 ml of acetone. The mixture is cooled to 0° C. in an ice bath and a solution of Jones reagent (pre-prepared by dissolving 26.7 g of CrO₃ in 23 ml of concentrated H₂SO₄ and then making the volume up to 100 ml with water) is added slowly until a persistent red-brown coloration is obtained. A few drops of isopropanol are added in order to decompose the excess chromium (VI). The mixture is taken up in 30 ml of a saturated aqueous NaCl solution and extracted with 4×50 ml of Et₂O. The organic phases are combined, washed with 30 ml of a saturated aqueous NaCl solution, dried over MgSO₄, and then filtered. The solvents are distilled off under reduced pressure, to give a yellow oil. The crude product is purified by silica chromatography (80/20 petroleum ether/diethyl ether), to give 1.34 g of trisnorsqualenic acid.

¹H NMR (CDCl₃, 300 MHz) δ: 5.19-5.07 (5H, m, vinyl CH); 2.45 (2H, t, J=7.3 Hz, CH₂CH₂COOH), 2.30 (2H, t, J=7.3 Hz, CH₂CH₂COOH), 2.09-1.98 (16H, m, allyl CH₂), 1.68 (3H, s, CH₃); 1.62 (3H, s, CH₃), 1.60 (12H, s, CH₃);

¹³C NMR (CDCl₃, 75 MHz), δ: 180.0 (CO), 135.0 (C), 134.8 (2 C), 132.8 (C), 131.1 (C), 125.3 (CH), 124.4 (2 CH), 124.2 (2 CH), 39.7 (2 CH₂), 39.5 (CH₂), 34.2 (CH₂) 33.0 (CH₂), 28.2 (2 CH₂), 26.8 (CH₂), 26.6 (2 CH₂), 25.6 (CH₃), 17.6 (CH₃), 16.0 (4 CH₃).

IR (cm⁻¹): 2966, 2916, 2857, 1709, 1441, 1383, 1299, 1212, 1155, 1103;

CIMS (isobutane) m/z 401 (100);

EIMS m/z 400 (5), 357 (3), 331 (5), 289 (3), 208 (6), 136 (3), 81 (100).

### b) Synthesis of (N)-squalenoylampicillin

The ampicillin was conjugated by condensation with the mixed anhydride, derived from 1,2,2′-trisnorsqualenic acid, formed in situ.

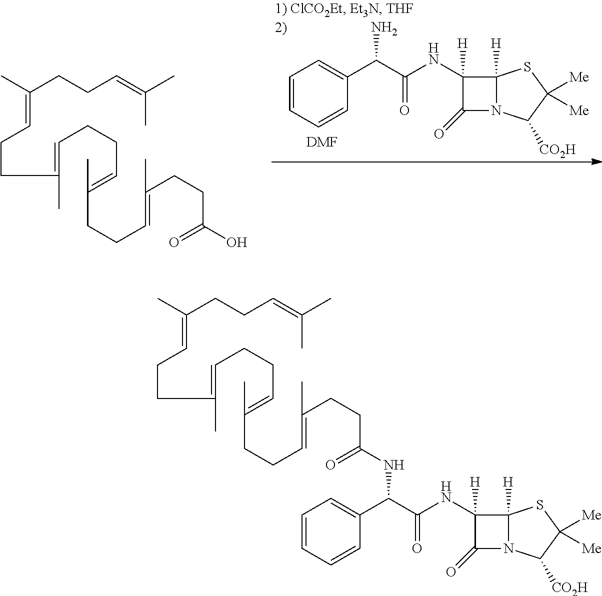

150 mg of triethylamine (1.5 mmol) and 120 mg of ethyl chloroformate (1.1 mmol) are added sequentially to a solution of 400 mg of 1,2,2′-trisnorsqualenic acid (1 mmol) in THF (4 ml) at 0° C. A white precipitate immediately forms. The mixture is stirred for 30 minutes at 0° C., and then a solution of ampicillin (440 mg; 1.3 mmol) and of 150 mg of triethylamine (1.5 mmol) in DMF (2 ml) is added dropwise. The mixture is stirred at 20° C. for 24 h and then the DMF is distilled off under reduced pressure. The residue is taken up with 0.5 N HCl until a pH of 2-3. The mixture is extracted with ethyl acetate (4×10 ml). The combined organic phases are washed with a saturated aqueous NaCl solution (1 ml), dried over MgSO₄, and concentrated under reduced pressure. The residue is taken up in 50 ml of ethyl acetate and washed in distilled water (3×1 ml). The organic phase is dried and concentrated under reduced pressure, to give 560 mg of ampicillin-squalene in the form of a pasty solid.

IR (pure, cm⁻¹) ν: 3400-3100, 1784, 1639, 1534, 1447, 1373, 1299, 1214; ¹H NMR (300 MHz, CDCl₃) δ: 7.40-7.20 (m, 5H); 7.01 (d, J=6.0 Hz, 1H); 6.95 (m, 1H); 5.64 (d, J=6.5 Hz, 1H); 5.57 (dd, J=8.5; 4.1 Hz, 1H); 5.42 (d, J=3.8 Hz, 1H); 5.20-5.05 (m, 5H); 4.35 (s, 1H); 2.40-2.20 (m, 4H); 2.10-1.90 (m, 16H); 1.67 (s, 3H); 1.59 (s, 12H); 1.57 (s, 3H); 1.54 (s, 3H); 1.49 (s, 3H);

MS (-APCI): m/z (%)=730 (100) [M-H]⁻.

### EXAMPLE 2

### Preparation of Nanoparticles of the Ampicillin-SQ

The nanoparticles are obtained by means of the precipitation/solvent evaporation method, by analogy with the method described in Fessi H. et al, Int. J. Pharm., 55; 1989, R1-R4.

A solution of 17 mg of squalenized ampicillin in ethanol (3 ml) is added dropwise to 4 ml of MilliQ® water, with magnetic stirring. The particles form instantaneously. After 2 or 3 minutes of stirring, the suspension of nanoparticles is transferred into a tared 100 ml round-bottomed flask and concentrated under reduced pressure in a rotary evaporator (50-100 mbar at 20° C. for 10 min then at 37° C. for approximately 3-5 minutes) until a weight of 3.7 g is obtained. The solution is then made up to 4 g using either MilliQ® or sterile water.

The size of the nanoparticles obtained, measured in a Malvern nanosizer (Zetasizer), is 169 nm.

The resulting nanoparticles have good stability in an aqueous solution (greater than 16 hours at 0° C.). They have a polydispersity index (PDI) of 0.076.

The polydispersity index was determined according to the methods well known to those skilled in the art.

### EXAMPLE 3

### Preparation of the (N)-squalenoylamoxicillin (SQ-amoxi) (or 3,3-dimethyl-7-oxo-6-[2-(4,8,13,17,21-pentamethyldocosa-4,8,12,16,20-pentaenoylamino)-2-(4-hydroxyphenyl)acetylamino]-4-thia-1-aza-bicyclo[3.2.0]heptane-2-carboxylic acid) complex

150 mg of triethylamine (1.5 mmol) and 120 mg of ethyl chloroformate (1.1 mmol) are added sequentially to a solution of 400 mg of 1,2,T-trisnorsqualenic acid as described in example 1.a (1 mmol) in THF (4 ml) at 0° C. A white precipitate forms immediately. The mixture is stirred for 30 minutes at 0° C., and then 2 ml of DMF and 150 mg of triethylamine (1.5 mmol) are added, followed by 474 mg of solid amoxicillin (1.3 mmol). The mixture is stirred at 20° C. for 48 h and then the DMF is distilled off under reduced pressure. The residue is taken up with 0.5 N HCl until a pH of 2-3. The mixture is extracted with ethyl acetate (4×15 ml). The combined organic phases are washed with a 0.05 N solution of HCl (3×2 ml), dried over MgSO₄ and concentrated under reduced pressure, to give 360 mg of amoxicillin-squalene in the form of an amorphous white solid.

IR (pure, cm⁻¹) ν: 3500-3100, 2977, 1778, 1641, 1613, 1514, 1448, 1384, 1268, 1211;

¹H NMR (300 MHz, acetone-d6) δ: 7.90 (d, J=8.7 Hz, 1H, NH), 7.58 (d, J=7.5 Hz, 1H, NH) 7.28 (d, J=8.4 Hz, 2H), 6.79 (d, J=8.4 Hz, 2H), 5.68-5.60 (m, 2H); 5.50 (d, J=4.2 Hz, 1H), 5.25-5.05 (m, 5H), 4.32 (s, 1H); 2.42-2.30 (m, 2H); 2.30-2.20 (m, 2H), 2.15-1.90 (m, 16H); 1.66 (s, 3H); 1.59 (s, 15H) 1.59 (s, 3H), 1.52 (s, 3H);

MS (-APCI): m/z (%)=746 (100) [M-H]⁻.

### EXAMPLE 4

### Preparation of Nanoparticles of the Amoxicillin-SQ

The nanoparticles are obtained by means of the precipitation/solvent evaporation method, by analogy with the method described in Fessi H. et al, Int. J. Pharm., 55; 1989, R1-R4.

A solution of 7.5 mg of squalenized amoxicillin in ethanol (1.0 ml) is added dropwise to 1.5 ml of MilliQ® water, with magnetic stirring. The particles form instantaneously. After 2 or 3 minutes of stirring, the suspension of nanoparticles is transferred into a tared 50 ml round-bottomed flask and concentrated under reduced pressure in a rotary evaporator (50-100 mbar at 20° C. for 10 mm and then at 37° C. for approximately 3-5 minutes) until a weight of 1.2 g is obtained. The solution is they made up to 1.5 g using either MilliQ® water or a sterile water.

The size of the nanoparticles obtained, measured in a Malvern nanosizer (Zetasizer), is 91 nm.

The resulting nanoparticles have good stability in an aqueous solution (greater than 16 hours at 0° C.). They have a polydispersity index (PDI) of 0.14.

The PDI was determined according to the methods well known to those skilled in the art (for example, by analogy with the method described in. Couvreur et al., Nanoletters, vol. 6, no. 11, pages 2544-2548, 2006).

### EXAMPLE 5

### Preparation of the 2-oxo-2-{[(4E,8E,12E,16E)-4,8,13,17,21-pentamethyldocosa-4,8,12,16,20-pentaen-1-yl]oxy}ethyl (2S,5R,6R)-3,3-dimethyl-7-oxo-6-[(phenylacetyl)amino]-4-thia-1-azabicyclo[3.2.0]heptane-2-carboxylate (also known as penicillin G-SQ or squalenoyl-penicillin G) complex

The preparation of the penicillin G-SQ complex can be represented schematically as follows.

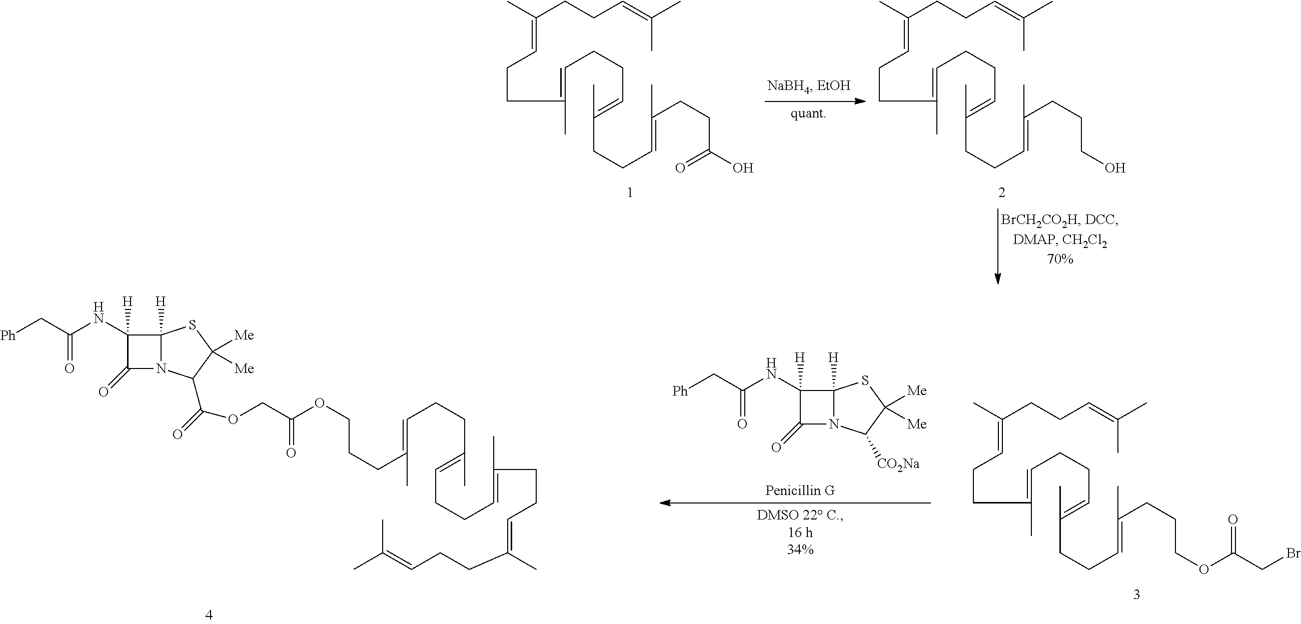

### a. Synthesis of trisnorsqualenyl bromoacetate (or (4E,8E,12E,16E)-4,8,13,17,21-pentamethyldocosa-4,8,12,16,20-pentaen-1-yl bromoacetate)

106 mg (0.9 eq., 2.7 mmol) of sodium borohydride are added, at a temperature of 0° C., in small portions, to 1.15 g (3 mmol) of 1,1′,2-trisnorsqualenic aldehyde (1) dissolved in 6 ml of ethanol.

The mixture is then stirred at ambient temperature for 15 min, and neutralized using a solution of HCl (1N), and then the solvent is distilled off under reduced pressure. The residue is taken up in 10 ml of water and extracted with ethyl acetate (3×20 ml). The combined organic phases are washed with a saturated aqueous solution of NaCl (10 ml), dried over MgSO₄ and filtered. The solvent is dissolved off under reduced pressure, to give a pale yellow oil (2).

IR (pure, cm⁻¹) ν: 3060-2840, 1686 (weak), 1449, 1381.

¹H NMR (300 MHz, CDCl₃) δ: 5.16-5.09 (m, 5H), 3.62 (t, J=6.4, 2H), 2.13-1.94 (m, 20H). 1.61 (s, 3H), 1.53 (s, 15H).

216 mg (1.55 eq., 2.01 mmol) of bromoacetic acid and a few mg of DMAP are added to 500 mg (1.3 mmol) of trisnorsqualene alcohol (2), obtained previously, dissolved in 6 ml of anhydrous CH₂Cl₂. The mixture is cooled to 0° C., and then 317 mg (1.5 equiv, 1.95 mmol) of DCC dissolved in 2 ml of CH₂Cl₂ are added in small portions. Once the addition is complete, the mixture is stirred under a nitrogen atmosphere at ambient temperature for 18 h, and then filtered through celite. The solvent is distilled off under reduced pressure. The residue is chromatographed on silica gel using an EtOAc/cyclohexane (1/4) mixture, to give 460 mg of a colorless oil (3).

IR (pure, cm⁻¹) ν: 2960-2850, 1739, 1700, 1448, 13821276, 1381; ¹H NMR (300 MHz, CDCl₃) δ: 5.18-5.07 (m, 5H), 4.14 (t, J=6.4, 2H), 3.82 (s, 2H), 2.16-1.98 (m, 18H), 1.81-1.71 (m, 2H), 1.68 (s, 3H), 1.53 (s, 15H).

¹³C NMR (75 MHz, CDCl₃) δ: 167.2 (C), 135.0 (C), 134.8 (2C), 133.2 (C), 131.2 (C), 125.3 (CH), 124.4 (2 CH), 124.2 (2 CH), 65.9 (CH₂), 39.7 (2 CH₂), 39.6 (CH₂), 35.5 (CH₂), 28.2 (2 CH₂), 27.8 (CH₂), 26.6 (CH₂), 26.5 (2 CH₂), 25.8 (CH₂), 25.6 (CH₃), 17.6 (CH₃), 16.0 (3 CH₃), 15.8 (CH₃),

### b) Synthesis of 2-oxo-2-{[(4E,8E,12E,16E)-4,8,13,17,21-pentamethyldocosa-4,8,12,16,20-pentaen-1-yl]oxy}ethyl (2S,5R,6R)-3,3-dimethyl-7-oxo-6-[(phenyl-acetyl)amino]-4-thia-1-azabicyclo[3.2.0]heptane-2-carboxylate

A mixture of 50 mg of penicillin G (0.15 mmol) and 113 mg of 1,1′,2-trisnorsqualenyl bromoacetate (3) (0.22 mmol) in anhydrous DMSO (0.75 ml) is stirred at 20° C. After 48 h, the mixture is concentrated under reduced pressure (0.05 Torr).

The residue is purified directly by silica gel chromatography, elution being carried out with an EtOAc/cyclohexane (1/4) mixture, to give 39 mg of penicillin G-squalene (4) in the form of a viscous liquid.

[α]_{D}=+213.3 (EtOH, c=0.45).

IR (pure, cm⁻¹) ν: 3400-3100, 2931, 2854, 1789, 1753, 1691, 1659, 1495, 1453, 1375, 1293, 1199, 1177, 1152.

¹H NMR (300 MHz, CDCl₃) δ: 7.42-7.20 (m, 5H), 6.07 (d, J=9.1 Hz, 1H, CONH), 5.66 (dd, J=9.0, 4.2 Hz, 1H, H-6), 5.50 (d, J=4.2 Hz, 1H, H-5), 5.20-5.10 (m, 5H, HC═C(Me)), 4.75 (d, J=15.7 Hz, 1H, OCH₂CO₂), 4.75 (d, J=15.7 Hz, 1H, OCH₂CO₂), 4.43 (s, 1H, H-2), 4.13 (t, J=6.7 Hz, 2H, CO₂CH₂CH₂), 3.61 (s, 2H, PhCH₂CO), 2.12-1.92 (m, 18H), 1.73 (q, J=8.0 Hz, 2H), 1.67 (s, 3H), 1.59 (s, 15H), 1.54 (s, 3H, SC(CH₃)₂), 1.50 (s, 3H, SC(CH₃)₂).

¹³C NMR (75 MHz, CDCl₃) δ: 173.7 (CO, C-7), 170.3 (CO, CO₂), 167.0 (CO), 166.9 (CO), 135.1 (C), 134.9 (2C), 133.8 (C), 133.2 (C), 131.2 (C), 129.5 (2CH), 129.1 (2CH), 127.6 (CH), 125.4 (CH), 124.4 (2 CH), 124.2 (2CH), 70.2 (CH, C-2), 67.9 (CH, C-5), 65.4 (CH₂, CO₂CH₂CH), 64.6 (C, C-3,), 61.2 (CH₂, OCH₂CO₂); 58.5 (CH, C-6), 43.3 (CH₂), 39.7 (2 CH₂), 39.6 (CH₂), 35.5 (CH₂), 31.1 (CH₃), 28.2 (3 CH₂), 26.7 (CH₃), 26.7 (CH₂), 26.6 (4 CH₂), 25.6 (CH₃), 17.6 (CH₃), 16.0 (3 CH₃), 15.8 (CH₃). MS (-APCI): m/z (%)=760 (100) [M-H]⁻¹.

### EXAMPLE 6

### Preparation of Nanoparticles of Penicillin G-SQ

The nanoparticles are obtained by means of the precipitation/solvent evaporation method, by analogy with the method described in H. Fessi et al, ibid. and the procedure described in example 2.

More particularly, a solution of 4 mg of squalenized penicillin G obtained in example 5, in ethanol (0.5 ml), is added dropwise to 1.0 ml of an aqueous solution of sucrose at 5%, with magnetic stirring. The particles form instantaneously. After 2 or 3 minutes of stirring, the suspension of nanoparticles is transferred into a tared 50 ml round-bottomed flask and concentrated under reduced pressure in a rotary evaporator (50-100 mbar at 20° C. for 10 min and then 37° C. for approximately 3-5 minutes) until a weight of 0.9 g is obtained. The solution is then made up to 1.0 g using an aqueous solution of sucrose at 5%.

The size of the nanoparticles obtained, measured in a Malvern nanosizer (Zetasizer), is 191 nm.

The resulting nanoparticles have good stability in an aqueous solution (greater than 24 h at 0° C.). They have a polydispersity index of 0.14, determined according to the methods well known to those skilled in the art.

1. A complex made up of at least one beta-lactam molecule covalently coupled to at least one hydrocarbon-based radical, said hydrocarbon-based radical being represented by the radical of formula (I) which follows: 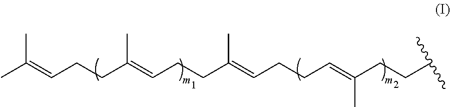
in which:
m₁=1, 2, 3, 4, 5 or 6;
m₂=0, 1, 2, 3, 4, 5 or 6; and 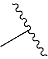
represents the bond toward the molecule, derived from beta-lactam, it being understood that, when m₂ represents 0, then m₁ represents at least 2.

2. The complex of claim 1, in which the hydrocarbon-based compound comprises from 18 to 40 carbon atoms.

3. The complex of claim 1, in which the hydrocarbon-based radical is the radical of formula (I) in which m₁ represents 1 and m₂ represents 2.

4. The complex of claim 1, in which the beta-lactam molecule is represented by formula (II): 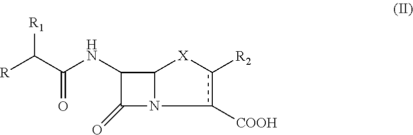
In which:
X represents a heteroatom chosen from a sulfur, an oxygen, a nitrogen or else an —S—CH₂—, —CH₂—S—, —CH₂— or —(CH₂)₂— group; [symbol]
indicates the optional presence of a double bond;
R represents an aryl, —O-phenyl or heteroaryl group, said groups being optionally substituted with one or more R₃ group(s);
R₃ represents a halogen atom, a hydroxyl group, a C₁-C₆ alkyl group, a C₁-C₆ alkoxy group, said alkyl and alkoxy groups being optionally substituted with one or more halogen atoms or with one or more hydroxyl groups, an —NR₄R₅ group, a —COOR₆ group or a —CONR₄R₅ group;
R₄ and R₅ represent, independently of one another, a halogen atom, or a C₁-C₆ alkyl group optionally substituted with one or more halogen atoms or with one or more hydroxyl groups;
R₆ represents a hydrogen atom, or a C₁-C₆ alkyl optionally substituted with one or more halogen atoms or with one or more hydroxyl groups;
R₁ represents a hydrogen, or a —COOR₆, —NR₄R₅ or ═N—OCH₃ group;
R₂ represents one or two group(s) independently chosen from a hydrogen atom, a halogen atom, a hydroxyl group, a C₁-C₆ alkyl and a C₁-C₆ alkoxy, said alkyl or alkoxy groups being optionally substituted with one or more halogen atoms or with one or more hydroxyl groups or with an —O—C(O)—C₁-C₆ alkyl group;
and the pharmaceutically acceptable salts thereof.

5. The complex of claim 1, in which the beta-lactam molecule is chosen from the family of penicillins, cephalosporins and carbapenems.

6. The complex of claim 1, in which the beta-lactam molecule is chosen from amoxicillin, ampicillin, penicillin G, cefotaxime, floxacillin, methicillin, dicloxacillin, carbenicillin and mezlocillin.

7. The complex of claim 1, in which the two entities forming said complex are coupled by means of a covalent bond of ester, ether, thioether, disulfide, phosphate or amide type.

8. The complex of claim 1, represented by the compound of formula (III) which follows: 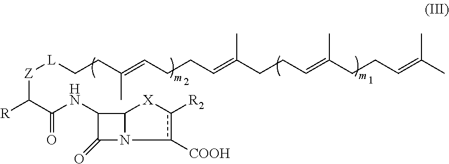
in which:
X, R and R₂ are as defined for the compound of formula (II) and m₁ and m₂ are as defined for the compound of formula (I); and
Z represents a covalent bond of ester, ether, thioether, disulfide, phosphate or amide type and L represents a single covalent bond or a C₁-C₄ alkylene group.

9. The complex of claim 1, characterized in that it has the ability to organize spontaneously in the form of nanoparticles when it is in the presence of an aqueous medium.

10. Nanoparticles of a complex as described in claim 1.

11. The nanoparticles of claim 10, the mean size of which ranges from 30 to 500 nm.

12. The nanoparticles of claim 10, chosen from the nanoparticles of (N)-squalenoyl-ampicillin, of (N)-squalenoyl-amoxicillin and of squalenoyl-penicillin G.

13. A method for preparing nanoparticles, characterized in that it comprises at least:
the dispersion of a complex of claim 1, in at least one organic solvent, at a concentration sufficient to obtain, when the resulting mixture is added, with stirring, to an aqueous phase, the instantaneous formation of nanoparticles of said complex in suspension in said aqueous phase, and, where appropriate, the isolation of said nanoparticles.

14. The preparation method of claim 13, also comprising a lyophilization step.

15. A lyophilisate comprising at least one complex as defined according to claim 1.

16. A pharmaceutical composition comprising at least one complex as defined according to claim 1, said complex being optionally in the form of a lyophilisate, in combination with at least one pharmaceutically acceptable vehicle.

17. The complex as defined according to claim 1, said complex being optionally in the form of a lyophilisate, for the treatment and/or prevention of bacterial infections, in particular caused by beta-lactam sensitive strains.
